(19) United States
(12) Patent Application Publication
(10) Pub. No.: US 2024/0264135 A1
(43) Pub. Date: Aug. 8, 2024

(54) METHOD AND SYSTEM FOR GAS DETECTION

(71) Applicant: Carrier Corporation, Palm Beach Gardens, FL (US)

(72) Inventors: Ashalatha Kapa, Hyderabad (IN); Srinivasa Reddy Pilli, Hyderabad (IN); Vinayak Rao Elur, Hyderabad (IN)

(21) Appl. No.: 18/432,582

(22) Filed: Feb. 5, 2024

Related U.S. Application Data
(60) Provisional application No. 63/483,433, filed on Feb. 6, 2023.

(51) Int. Cl. G01N 33/00; F24F 11/63; F24F 11/88; F24F 13/30

(52) U.S. Cl. CPC G01N 33/0031; F24F 11/63; F24F 11/88; F24F 13/30; G01N 33/0062

(57) ABSTRACT

Disclosed herein is a multi gas detection system comprising one or more sensors for detecting the presence of two or more of a refrigerant, a supply gas, and a combustion gas. Also disclosed is an HVAC system comprising the multi gas detection system and a method of operating the HVAC system.

### CROSS REFERENCE TO RELATED APPLICATIONS

This application claims the benefit of U.S. Provisional Patent Application No. 63/483,433 filed on Feb. 6, 2023, which is incorporated by reference herein in its entirety.

### BACKGROUND

Exemplary embodiments pertain to the art of gas monitoring, and more specifically to detection of gases in heating, ventilation, and air conditioning (HVAC) systems.

Gas sensors have been used in various applications such as process monitoring and control and safety monitoring. As the compounds can also be flammable or explosive, gas detection sensors have also been used for leak detection where such compounds are used or manufactured. There are viable sensor technology options, including, but not limited to, metal oxide semiconductor (MOS) sensors, non-dispersive infrared detector (NDIR) sensors, pellistor (pelletized resistor) sensors, ultrasonic sensors, high-temperature solid electrolytes that are permeable to oxygen ions, and electrochemical cells.

As use of low global warming potential (GWP) refrigerants expands there is an increasing need for sensors that can detect them.

### BRIEF DESCRIPTION

Disclosed herein is a multi-gas detection system comprising one or more sensors for detecting the presence of two or more of a refrigerant, a supply gas, and a combustion gas.

In any one or a combination of the foregoing embodiments, the multi gas detection system comprises a sensor component and the sensor component consists of one sensor.

In any one or a combination of the foregoing embodiments, the one or more sensors can detect an A2L refrigerant, a supply gas and a combustion gas.

In any one or a combination of the foregoing embodiments, the one or more sensors has a threshold limit of greater than or equal to 25% of the lower flammability limit for a refrigerant and a threshold limit of 1000 ppm for a supply gas, or a combination thereof.

In any one or a combination of the foregoing embodiments, the one or more sensors comprise an infrared sensor, an electrochemical cell sensor, a metal oxide semiconductor sensor, a pellistor, a heated diode sensor, a molecular property sensor, or a combination thereof.

Also disclosed herein is an HVAC system comprising a fan; a first heat exchanger located in an airflow created by the fan; a second heat exchanger located in the airflow created by the fan and downstream of the first heat exchanger; and multi gas detection system located between the first heat exchanger and the second heat exchanger wherein the multi gas detection system comprises one or more sensors for detecting the presence of two or more of a refrigerant, a supply gas, and a combustion gas.

In any one or a combination of the foregoing embodiments, the multi gas detection system comprises a sensor component and the sensor component consists of one sensor.

In any one or a combination of the foregoing embodiments, the one or more sensors can detect an A2L refrigerant, a supply gas and a combustion gas.

In any one or a combination of the foregoing embodiments, the one or more sensors has a threshold limit of greater than or equal to 25% of the lower flammability limit for a refrigerant and a threshold limit of 1000 ppm for a supply gas, or a combination thereof.

In any one or a combination of the foregoing embodiments, the one or more sensors comprise an infrared sensor, an electrochemical cell sensor, a metal oxide semiconductor sensor, a pellistor, a heated diode sensor, a molecular property sensor, or a combination thereof.

Also disclosed herein is a method of operating an HVAC system comprising monitoring an air flow for one or more of a refrigerant gas, a supply gas, and a combustion gas using a multi gas detection system; sending a signal to a controller when an amount of one or more of the refrigerant gas, supply gas, and combustion gas are above a threshold, wherein the signal includes the identity of the gas over the threshold; and initiating a mitigating response based on the identity of the gas over threshold.

In any one or a combination of the foregoing embodiments, the gas over the threshold is a refrigerant gas and the mitigating response comprises halting operation of the system, turning on a blower fan and opening a zoning damper.

In any one or a combination of the foregoing embodiments, the gas over the threshold is a supply gas and the mitigating response comprises halting operation of the system, turning on a blower fan and opening a zoning damper.

In any one or a combination of the foregoing embodiments, the gas over the threshold is a combustion gas and the mitigating response comprises halting operation of the system and turning off a blower fan.

### BRIEF DESCRIPTION OF THE DRAWINGS

The following descriptions should not be considered limiting in any way. With reference to the accompanying drawings, like elements are numbered alike:

FIG. 1 is an illustration of a residential heating and cooling system;

FIG. 2 is a flow chart of a logic routine for a mitigating response after gas detection.

FIG. 3 is an illustration of a residential heating and cooling system showing the dampers.

### DETAILED DESCRIPTION

A detailed description of one or more embodiments of the disclosed apparatus and method are presented herein by way of exemplification and not limitation with reference to the Figures.

As the use of low global warming refrigerants expands there is an increasing need for sensors that can detect them. Low global warming potential (GWP) refrigerants can be mildly flammable. Additionally, there is an increasing need for sensors that can also detect combustion gas leakage and supply gas leakage. Air conditioning and heating systems may be combined for efficiency and sustainability with a common heat exchanger system used for cooling and heating. Heating may employ combustible gasses and supply gasses as a source of heat. Currently there is a need for a detection system which is capable of detecting two or more target gasses such as refrigerant gasses, combustion gasses and supply gasses. There is a further need for an HVAC system and method of operating the HVAC system which can initiate mitigation actions appropriate to the detected target gas.

The detection system disclosed herein includes one or more sensors which detect the presence of two or more target gasses. Target gasses include a refrigerant gas, a combustion product gas, and a supply gas. In some embodiments a single sensor may be able to detect two or more types of gasses, for example, a refrigerant gas and a supply gas. In some embodiments a single sensor may be able to detect all three types of gasses. In some embodiments the detection system may include a sensor for each type of gas—refrigerant, supply, and combustion. The detection system may include a processor which analyzes the data from the sensor(s), determines the concentration of the target gas and determines whether a target gas is present in an amount above a threshold and transmits that information to a controller for the HVAC system. The controller initiates mitigation actions when a target gas is present in an amount above a threshold as explained below. Alternatively, the detection system may transmit the data to a controller for the HVAC system. The controller then analyzes the data and determines the concentration of the target gas and whether the target gas is present in an amount above a threshold and initiates mitigation actions when the target gas is present in an amount above a threshold amount.

Exemplary refrigerant gasses include A3 refrigerants, A2L refrigerants, and A2/A2L blends. This classification of refrigerants is based on the safety classification by ASHRAE (American Society of Heating, Refrigerating and Air-Conditioning Engineers). The classification system is shown below. LFL is the abbreviation for lower flammability limit. ETFL₆₀ is the abbreviation for elevated temperature flame limit at 60° C. HOC is the abbreviation for heat of combustion. OEL is the abbreviation for occupational exposure limit.

| Flammability in Air @ 60° C. & 101.3 kPa | Safety group | |
|---|---|---|
| Higher Flammability | A3 | B3 |
| LFL or ETFL₆₀ = 100 g/m³ OR HOC = 19 | | |
| MJ/kg | | |
| Lower Flammability | A2 | B2 |
| LFL or ETFL₆₀ > 100 g/m³ & HOC < 19 | | |
| MJ/kg] | | |
| Lower Flammability | A2L | B2L |
| LFL or ETFL₆₀ > 100 g/m³ & HOC < 19 | | |
| MJ/k with a maximum burning velocity | | |
| of = 10 cm/s | | |
| No flame Propagation | A1 | B1 |
| Flammability in Air @ 60° C. & 101.3 kPa | Lower | Higher |
| | Toxicity | Toxicity |
| | OEL ≥400 | OEL <400 |
| | ppm | ppm |

A3 refrigerants include R-290 (propane), R-600 (butane), R-600a (isobutane) and combinations thereof. A2L refrigerants include R-1234yf, R-1234ze, R-32, R454A, R-454B, R-454C, R-455A, R-447A, R-452B, and combinations thereof.

Exemplary supply gasses include methane and propane. As appreciated by one of skill in the art, propane may be both supply gas and refrigerant.

Exemplary combustion gasses include NOₓ gasses and carbon monoxide. NOₓ gasses include N₂O₄, N₂O, NO, N₂O₃, NO₂, N₂O₅, N₂O₂, and combinations thereof.

Exemplary types of sensors include infrared (IR) such as non-dispersive IR (NDIR) and photoacoustic IR (PIR), electrochemical, metal oxide semiconductor (MOS), catalytic (pellistor), heated diode, and molecular property spectrometer sensors.

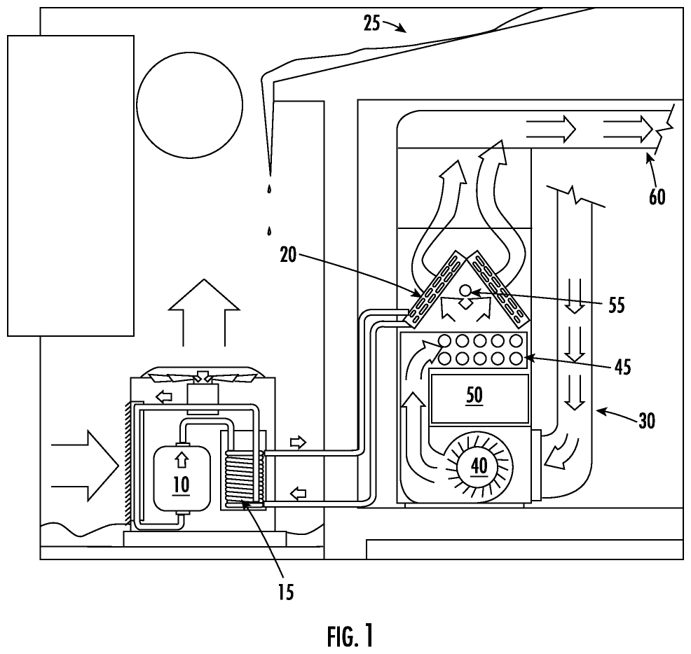

An example embodiment of a heat transfer system (also referred to as an HVAC system) operating as a heat pump is shown in FIG. 1. As shown in FIG. 1, a HVAC system includes a compressor 10 which pressurizes the refrigerant in its gaseous state, which both heats the fluid and provides pressure to circulate it throughout the system. When operating in heat pump mode, hot pressurized gaseous heat transfer fluid exiting from the compressor 10 flows through reversing valve 15 to heat exchanger 20 located inside of structure 25. Air from inside of structure 25 flows through return duct 30 drawn by fan 40 and passes through heat exchanger 45 and heat exchanger 20, returning to the inside of the structure through supply duct 60. Heat exchanger 45 is heated by combustion furnace 50. One or more sensors 55 for the multi gas detection system is located between heat exchanger 20 and heat exchanger 45.

The one or more sensor 55 can be operated to measure a primary parameter indicative of presence of a flammable gas. The multi gas detection system may further include other sensing element(s) to measure a secondary parameter(s) such as temperature, and/or humidity, and/or gas flow in the enclosed space. In the event of an actual leak of flammable vapor, physical changes to the surrounding space such as changes in temperature, and/or humidity, and/or gas flow are believed to result from the leakage and vaporization of refrigerant from the system. More specifically, leaking refrigerant in a substantial rate can cause temperature to drop, relative humidity to drop or both, compared to normal conditions. Although these parameters may not be dispositive by themselves of a flammable gas leak, they can be used to identify false positive alarms from the primary sensor such as a MOS or ultrasonic sensor by comparing observed measurements to normal measurements or to criteria indicative of a gas leak during a time period proximate to the positive signal produced by the primary sensor.

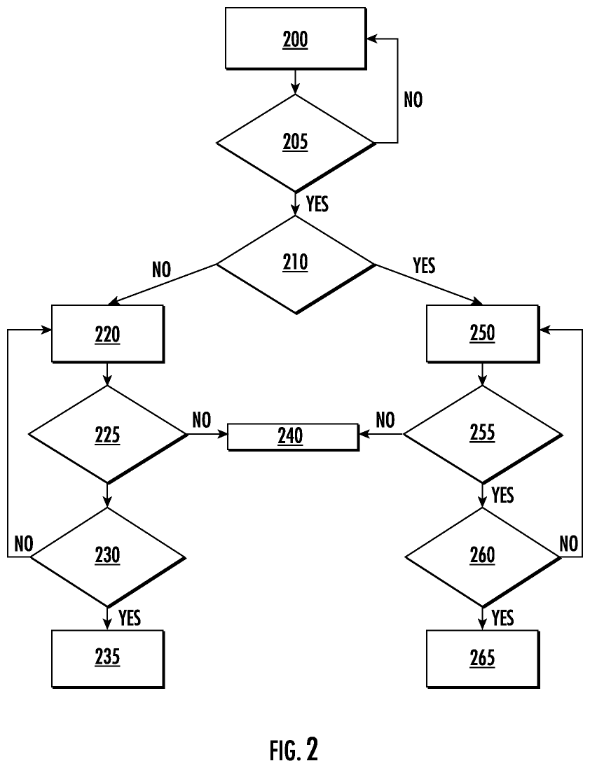

A protocol for operating an HVAC system with a multi gas detection system is shown in FIG. 2. In the flow chart, box 200 represents a condition where the heat pump or gas furnace is on, and the multi gas detection system is on. The one or more sensors of the multi gas detection system transmit a signal to a processor that is either part of the multi gas detection system or the HVAC system controller. The processor analyzes the signal from the one or more sensors and determines if a target gas is present in an amount greater than a threshold amount. In decision box 205 if a target gas amount is not greater than the threshold monitoring continues. If a target gas amount is greater than a threshold the type of target gas above the threshold is identified as shown in decision box 210. At decision box 210 the mitigation pathway is decided based on the type of target gas above the threshold, i.e., whether the target gas is a combustion gas.

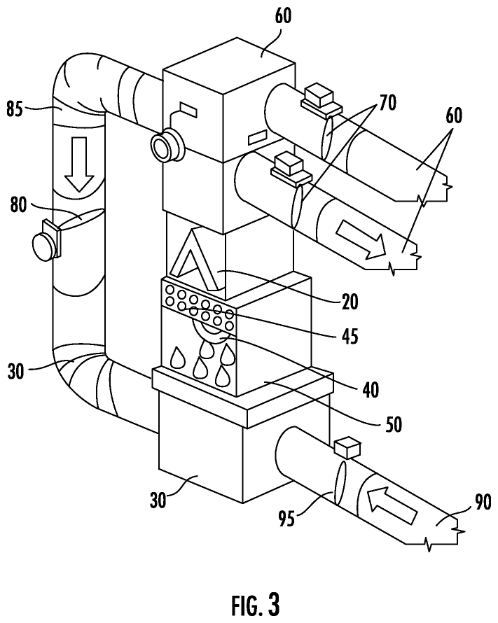

If the target gas present in an amount greater than a threshold is not a combustion gas (i.e., the target gas present in an amount greater than a threshold is a refrigerant gas, supply gas or combination thereof) the protocol proceeds to mitigation (box 220). Mitigation includes halting operation of the system. The threshold for a refrigerant gas is a concentration greater than or equal to 25% of the lower flammability limit determined by ASHRAE. The threshold for a supply gas is a threshold limit value (TLV) greater than or equal to 1000 parts per million (ppm) (limit determined by the Occupational Safety and Health Administration (OSHA) and cited by the American Conference of Governmental Industrial Hygienists (ACGIH)). Mitigation actions further include turning on the blower fan 40 and opening at least one zoning damper 70 and optionally a short return or any external zoning duct dampers 80. Additionally mitigation can include activating additional mechanical ventilation. The zoning damper and short return duct dampers or bypass damper are shown in FIG. 3. Duct damper 80 is disposed in duct 85 which is depicted as a short return or bypass duct in FIG. 3 but may intersect with another return duct from a zone. FIG. 3 also includes a fresh air duct 90 and a fresh air duct damper 95.

The signals from the one or more sensors of the multi gas detection system continue to be analyzed. At decision box 225 if the amount of the target gas (or gasses) decreases then the protocol proceeds to decision box 230. At decision box 230 the sensor values are again compared to the threshold values. If the sensor values are not below the threshold, then mitigation actions are continued. If the sensor values are below the threshold, then the protocol (method) optionally continues mitigation for a desired period of time and then halts the mitigation actions and returns to normal operation and monitoring (box 235). It is also contemplated that mitigation actions may be immediately halted, the system is restarted, and routine monitoring started again (box 235).

If the amount of target gas (or gasses) does not decrease, then a mitigation fault alarm is triggered (box 240) and the user is notified to call for service or other appropriate action.

If the target gas present in an amount greater than a threshold is a combustion gas the protocol proceeds to mitigation (box 250). Mitigation includes halting operation of the system. The user may be notified. The threshold for a nitrogen containing combustion gas is a TLV greater than or equal to 3 parts per million (ppm). The threshold for carbon monoxide is a TLV greater than or equal to 25 ppm. Mitigation actions include turning off the blower fan (40). The signals from the one or more sensors of the multi gas detection system continue to be analyzed. At decision box 255 if the amount of the target gas (or gasses) decreases the protocol (method) proceeds to box 260. At decision box 260 if the combustion gas amount is less than the threshold, mitigation may be halted or mitigation may be continued for a defined period of time, such as five minutes, and then halted. After mitigation is ended (box 265) routing operation and monitoring is started. At decision box 260 if the amount of target gas (or gasses) is above the threshold amount the mitigation actions are continued until the amount of target gas (or gasses) is below the threshold amount.

If the amount of target gas (or gasses) does not decrease, then a mitigation fault alarm is triggered (box 240) and the user is notified to call for service or other appropriate action.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made, and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure is not limited to the particular embodiment disclosed as the best mode contemplated for carrying out this present disclosure, but that the present disclosure will include all embodiments falling within the scope of the claims.

What is claimed is:

1. A multi gas detection system comprising one or more sensors for detecting the presence of two or more of a refrigerant, a supply gas, and a combustion gas.

2. The multi gas detection system of claim 1, wherein the multi gas detection system comprises a sensor component and the sensor component consists of one sensor.

3. The multi gas detection system of claim 1, wherein the one or more sensors can detect an A2L refrigerant, a supply gas and a combustion gas.

4. The multi gas detection system of claim 1, wherein the one or more sensors has a threshold limit of greater than or equal to 25% of the lower flammability limit for a refrigerant and a threshold limit of 1000 ppm for a supply gas, or a combination thereof.

5. The multi gas detection system of claim 1, wherein the one or more sensors comprise an infrared sensor, an electrochemical cell sensor, a metal oxide semiconductor sensor, a pellistor, a heated diode sensor, a molecular property sensor, or a combination thereof.

6. An HVAC system comprising
a fan;
a first heat exchanger located in an airflow created by the fan;
a second heat exchanger located in the airflow created by the fan and downstream of the first heat exchanger; and
multi gas detection system located between the first heat exchanger and the second heat exchanger wherein the multi gas detection system comprises one or more sensors for detecting the presence of two or more of a refrigerant, a supply gas, and a combustion gas.

7. The HVAC system of claim 6, wherein the multi gas detection system comprises a sensor component and the sensor component consists of one sensor.

8. The HVAC system of claim 6, wherein the one or more sensors can detect an A2L refrigerant, a supply gas and a combustion gas.

9. The HVAC system of claim 6, wherein the one or more sensors has a threshold limit of greater than or equal to 25% of the lower flammability limit for a refrigerant and a threshold limit of 1000 ppm for a supply gas, or a combination thereof.

10. The HVAC system of claim 6, wherein the one or more sensors comprise an infrared sensor, an electrochemical cell sensor, a metal oxide semiconductor sensor, a pellistor, a heated diode sensor, a molecular property sensor, or a combination thereof.

11. A method of operating an HVAC system comprising
monitoring an air flow for one or more of a refrigerant gas, a supply gas, and a combustion gas using a multi gas detection system;
sending a signal to a controller when an amount of one or more of the refrigerant gas, supply gas, and combustion gas are above a threshold, wherein the signal includes the identity of the gas over the threshold; and
initiating a mitigating response based on the identity of the gas over threshold.

12. The method of claim 11, wherein the gas over the threshold is a refrigerant gas and the mitigating response comprises halting operation of the system, turning on a blower fan and opening a zoning damper.

13. The method of claim 11, wherein the gas over the threshold is a supply gas and the mitigating response comprises halting operation of the system, turning on a blower fan and opening a zoning damper.

14. The method of claim 11, wherein the gas over the threshold is a combustion gas and the mitigating response comprises halting operation of the system and turning off a blower fan.
